(19) United States
(12) Patent Application Publication
(10) Pub. No.: US 2026/0199210 A1
(43) Pub. Date: Jul. 16, 2026

(54) COSMETIC COMPOSITION COMPRISING 2,4-DIAMINOPYRIMIDINE 3-N-OXIDE, PIROCTONE OLAMINE, NIACINAMIDE, AND AT LEAST 1% BY WEIGHT OF PARTICULAR HYDROXYLATED COMPOUND

(71) Applicant: L'OREAL, PARIS (FR)

(72) Inventors: Christophe BOULLE, SAINT OUEN (FR); Olivia TOURIGUINE, SAINT OUEN (FR); Mathilde RIVIERE, SAINT OUEN (FR)

(21) Appl. No.: 19/139,122

(22) PCT Filed: Dec. 13, 2023
(86) PCT No.: PCT/EP2023/085486

§ 371 Date: Jun. 13, 2025

(30) Foreign Application Priority Data
Dec. 16, 2022 (FR) FR2213539

(51) Int. Cl. A61K 8/49; A61K 8/67; A61K 8/9794; A61Q 5/00

(52) U.S. Cl. CPC A61K 8/4953; A61K 8/4926; A61K 8/675; A61K 8/9794; A61Q 5/002; A61K 2800/48

(57) ABSTRACT

The present invention relates to a cosmetic composition comprising 2,4-diaminopyrimidine 3-N-oxide and/or an organic or mineral acid salt thereof and/or a hydrate thereof, piroctone olamine, niacinamide, and at least 1% by weight of particular hydroxylated compound.

The invention also relates to a process for the cosmetic treatment of human keratin materials such as the skin, notably human scalp, hair and/or eyelashes, comprising at least the application of the cosmetic composition according to the present invention.

The present invention also relates to the use of said cosmetic composition for treating the loss and/or promoting the regrowth of keratin fibres.

The present invention relates to a cosmetic composition comprising 2,4-diaminopyrimidine 3-N-oxide and/or an organic or mineral acid salt thereof and/or a hydrate thereof, piroctone olamine, niacinamide, and at least 1% by weight of particular hydroxylated compound.

The invention also relates to a process for the cosmetic treatment of human keratin materials such as the skin, notably human scalp, hair and/or eyelashes, comprising at least the application of the cosmetic composition according to the present invention.

The present invention also relates to the use of said cosmetic composition for treating the loss and/or promoting the regrowth of keratin fibres.

The present invention relates to the field of the cosmetic treatment of human keratin materials, in particular human keratin fibres, such as human head hair, eyebrows, eyelashes and/or bodily hair, notably beard hair, moustache hair and pubic hair, and of the skin, notably the scalp and areas of the skin, in particular alopecic or non-alopecic areas, preferably alopecic areas, covered with keratin fibres as mentioned previously. More particularly, the invention relates to the cosmetic treatment of human hair and/or eyelashes and/or skin, preferably hair and/or scalp.

The growth and renewal of the hair in humans are mainly determined by the activity of the hair follicles and of their dermo-epidermal environment. Their activity is cyclical and essentially comprises three phases, namely the anagenic phase, the catagenic phase and the telogenic phase.

The anagenic phase (corresponding to the active or growth phase), which lasts several years and during which the hairs lengthen, is followed by a very short and transient catagenic phase which lasts a few weeks. During this phase, the hair strand undergoes a change, the follicle atrophies and its dermal implantation appears higher and higher.

The terminal phase, which lasts a few months, corresponds to a resting phase known as the telogenic phase. At the end of this resting period, the hair falls out and is replaced by new hair follicles in the anagenic phase, in such a way that another hair cycle begins.

Head hair is thus constantly being renewed and, of the approximately 150 000 individual hairs which make up a head of hair, at any one moment approximately 10% are at rest and will thus be replaced in the months to come.

Alopecia denotes accelerated loss of head hair, eyelashes, eyebrows and/or bodily hair in any part of the body. It generally occurs in genetically predisposed individuals and mainly affects men. This is known as androgenetic or androgenic alopecia, or even androgeno-genetic alopecia.

Alopecia is essentially due to a disturbance in hair renewal, which initially leads to an acceleration in the frequency of the cycles at the expense of the hair quality and then its quantity. This results in a gradual impoverishment of the hair by the regression of the “terminal” hairs to the downy stage. Certain areas are preferentially affected: notably in men, the temporal or frontal lobes and also the upper part of the occipital lobe, whereas in women diffuse alopecia of the vertex is seen.

Other causes may bring about substantial temporary or permanent hair loss. These may include hair loss and impairments after pregnancy (or post partum), during malnutrition or dietary imbalances, or even asthenia or hormonal dysfunction, as may be the case during or after the menopause. It may also be a case of hair loss or impairments related to seasonal phenomena.

Alopecia may also affect the eyelashes, eyebrows and/or bodily hair on any part of the human body, and may cause aesthetic displeasure in the same way as alopecia affecting one or more areas of the scalp.

People who are thus prone to alopecia are increasingly resorting to cosmetic treatments that enable them to eliminate and/or reduce the effect of the alopecia, and notably to reduce or delay the loss of hair, eyelashes and/or bodily hair, and also to induce or stimulate their growth.

Such treatments generally consist of daily applications to the various alopecic areas of the human body, in particular to alopecic areas of the scalp, such as the temporal or frontal lobes in men, or areas of the body free of hair, in particular on the face, notably the beard, moustache and/or eyebrows, of cosmetic compositions comprising one or more agents for combating hair loss and/or for promoting hair regrowth.

To this end, agents such as minoxidil, finasteride, aminexil or stemoxydine may notably be mentioned as agents that are capable of reducing the loss and/or promoting the regrowth of keratin fibres.

The duration of these treatments generally ranges from several weeks to several months, and may even extend over years, and their long-term efficacy most often depends on assiduous regular application by the user of the cosmetic compositions to the areas to be treated, which may be more or less extensive.

The application of treatments that are capable of reducing the loss and/or promoting the regrowth of head hair, eyelashes and/or bodily hair may notably last several weeks before the desired effects can begin to be seen, in particular curbing alopecia.

These treatments must be used daily to obtain pronounced efficacy.

However, these compositions still too often have numerous application drawbacks which may have a negative impact on the cosmetic treatment to be followed, notably leading to more or less rapid abandonment of the treatment and, consequently, to poor results in terms of limiting or even eliminating alopecia.

Specifically, many compositions include gelling agents in order to prevent the composition from running onto the face, into the eyes or down the nape of the neck. However, the use of gelling agents, notably in the context of daily use, leads to aggregation of the keratin fibres, giving the head of hair a tacky effect that is unacceptable to consumers.

In order to improve the efficacy of these treatments, it may be necessary to increase the amounts of active agents, which may lead to difficulties in dissolving the active agents and to the formation of powders that are visible on the keratin fibres and scalp during use. This powdery effect is not acceptable to consumers either. Lotions for combating hair loss must be used daily to obtain pronounced efficacy. Some consumers wash their hair every day, in which case the products previously applied to the scalp are eliminated. Most consumers do not wash their hair every day, and so the product applied on the second day is added to the product applied on the first day. This is known as a cumulative effect. In this context of repeated application without washing the scalp, the appearance of powder may be present from the first day, and usually on the second day. This appearance of powder results from the precipitation of the solid compounds in the formula when the solvent evaporates.

As a result, the insufficient application qualities of these compositions may in the long term discourage users from assiduously following cosmetic treatments for combating hair loss and/or promoting hair regrowth, all the more so as the efficacy of these treatments is mainly linked to the regular daily application of the compositions described previously to the areas to be treated for several weeks or even several months.

In other words, a lack of regularity or abandonment of the treatment too quickly usually leads to poor results in terms of efficacy in combating hair loss and/or promoting hair regrowth and, consequently, to dissatisfaction. This leads to further disappointment among consumers, who do not have the time to observe the long-term progress and/or efficacy of these treatments.

There is thus a real need to overcome the drawbacks mentioned previously, notably by using a composition that is capable of cosmetically and effectively treating the loss of human keratin fibres such as human head hair, bodily hair and/or eyelashes, and/or of promoting their regrowth, which has improved application qualities while at the same time notably affording a satisfactory visual result, notably without a greasy effect and without residue. This composition must also allow repeated application, i.e. allow daily use of the composition.

This aim is achieved by the present invention, the subject of which is notably a cosmetic composition comprising:
i) 2,4-diaminopyrimidine 3-N-oxide and/or an organic or mineral acid salt thereof and/or a hydrate thereof,
ii) piroctone olamine,
iii) niacinamide, and
iv) at least 1% by weight, relative to the total weight of the composition, of one or more hydroxylated compounds with a melting point below 25° C. at atmospheric pressure (1.013×10⁵ Pa) (e.g. they are liquid at 25° C. and atmospheric pressure) and a boiling point of greater than or equal to 170° C., preferably chosen from hydroxylated compounds consisting of a C2-C8 hydrocarbon-based chain optionally interrupted with an oxygen atom and comprising from one to two free hydroxyl groups (—OH) borne by different carbon atoms.

The cosmetic composition according to the invention has improved application qualities by avoiding the powdery effect and/or aggregation effect on keratin fibres, in particular in the context of repeated application required for frequent use.

The cosmetic composition according to the invention is also entirely satisfactory in terms of its efficacy in combating the loss and/or promoting the regrowth of keratin fibres. It also has a beneficial effect on increasing the diameter of the keratin fibres and on strengthening the fibres against breakage.

In addition, the composition according to the invention gives good cosmetic properties, notably conditioning properties, to keratin fibres, particularly in terms of suppleness, disentanglement and a smooth feel. It also allows easy disentangling.

The invention also relates to a process for the cosmetic treatment of human keratin materials such as the skin, notably human scalp, hair and/or eyelashes, comprising at least the application of the cosmetic composition according to the present invention.

The present invention also relates to the use of said cosmetic composition for treating the loss and/or promoting the regrowth of keratin fibres.

The present invention also relates to the use of said cosmetic composition for increasing the diameter of keratin fibres and/or for reinforcing keratin fibres against breakage.

Other subjects, characteristics, aspects and advantages of the invention will emerge even more clearly on reading the description and the example that follows.

In the text hereinbelow, unless otherwise indicated, the limits of a range of values are included in that range, notably in the expressions “between” and “ranging from . . . to . . . ”.

Furthermore, the expression “at least one” used in the present description is equivalent to the expression “one or more”.

i) 2,4-diaminopyrimidine 3-N-oxide

The cosmetic composition according to the invention comprises 2,4-diaminopyrimidine 3-N-oxide and/or an organic or mineral acid salt thereof and/or a hydrate thereof.

2,4-Diaminopyrimidine 3-N-oxide has the following formula:

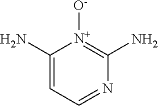

Among the organic or mineral acid salts that may be used, mention may be made in particular of the hydrochlorides, sulfates, citrates and acetates. As hydrate, mention may be made in particular of the monohydrate.

Preferably, the non-salified compound in monohydrate form is used.

Preferably, the total content of 2,4-diaminopyrimidine 3-N-oxide and/or an organic or mineral acid salt thereof and/or a hydrate thereof ranges from 0.05% to 8% by weight, better still from 0.1% to 5%, even better still from 0.2 to 4%, then from 0.5% to 3%, and preferentially from 1% to 2% by weight, relative to the total weight of the composition.

Preferably, the content of 2,4-diaminopyrimidine 3-N-oxide and/or a hydrate thereof, notably a monohydrate, ranges from 0.05% to 8% by weight, better still from 0.1% to 5%, even better still from 0.2% to 4%, from 0.5% to 3% and preferentially from 1% to 2% by weight, relative to the total weight of the composition.

### ii) Piroctone Olamine

The cosmetic composition according to the invention also comprises piroctone olamine.

Piroctone olamine is a monoethanolamine salt of 1-hydroxy-4-methyl-6-(2,4,4-trimethylpentyl)-2 (1H)-pyridinone.

Preferably, the total content of piroctone olamine ranges from 0.05% to 2% by weight, better still from 0.1% to 1%, preferentially from 0.1% to 0.5% by weight, relative to the total weight of the composition.

iii) Niacinamide

The cosmetic composition according to the invention also comprises niacinamide (3-pyridinecarboxamide).

Niacinamide is also known as nicotinamide.

Preferably, the total content of niacinamide ranges from 1% to 5% by weight, preferentially from 2% to 5% by weight, relative to the total weight of the composition.

### iv) Hydroxylated Compounds

The cosmetic composition according to the invention also comprises one or more hydroxylated compounds iv) with a melting point below 25° C. at atmospheric pressure (1.013×10⁵ Pa) (e.g. they are liquid at 25° C. and atmospheric pressure) and a boiling point of greater than or equal to 170° C.

Preferably, said hydroxylated compounds iv) consist of a C2-C8 hydrocarbon-based chain optionally interrupted with an oxygen atom and comprising from one to two free hydroxyl groups (—OH) borne by different carbon atoms.

These compounds may be cyclic or acyclic, linear or branched, saturated or unsaturated.

Preferably, the hydroxylated compound(s) iv) are chosen from linear C2-C8, better still C2-C6, diols or aromatic C6-C8, better still C7-C8, monoalcohols and mixtures thereof. More preferentially, the hydroxylated compound(s) iv) are chosen from linear C2-C8, preferably C2-C6, diols.

Mention may be made in particular of propylene glycol, butylene glycol, pentylene glycol, hexylene glycol, 1,3-propanediol, benzyl alcohol, phenethyl alcohol (phenylethyl alcohol) and dipropylene glycol, and even more preferentially the hydroxylated compound iv) is propylene glycol.

The total content of hydroxylated compound(s) iv) in the composition is greater than or equal to 1% by weight, relative to the total weight of the composition, preferably greater than or equal to 1.5% by weight, relative to the total weight of the composition.

Preferably, the total content of hydroxylated compounds iv) ranges from 1% to 15% by weight, better still from 1.5% to 10%, more preferentially from 2% to 8% by weight, even more preferentially from 2.1% to 6% by weight, better still from 2.2% to 5%, even better still from 2.3% to 4% by weight, relative to the total weight of the composition.

Preferably, the total content of hydroxylated compounds iv) chosen from linear C2-C8 diols is greater than or equal to 1% by weight, better still ranges from 1% to 15% by weight, preferentially ranges from 1.5% to 10% by weight, more preferentially from 2% to 8% by weight, even more preferentially from 2.1% to 6% by weight, better still from 2.2% to 5% by weight, even better still from 2.3% to 4% by weight, relative to the total weight of the composition.

Preferably, the total content of propylene glycol is greater than or equal to 1% by weight, better still ranges from 1% to 15% by weight, preferentially from 1.5% to 10% by weight, more preferentially from 2% to 8% by weight, even more preferentially from 2.1% to 6% by weight, better still from 2.2% to 5% by weight, even better still from 2.3% to 4% by weight, relative to the total weight of the composition.

### v) Ginger Root Extract

The cosmetic composition according to the invention may optionally also comprise a ginger root extract (INCI name: Zingiber officinale root extract).

The ginger root extract may be in crushed, powdered or liquid form, preferably in liquid form.

The ginger root extract may notably be obtained by supercritical CO₂ extraction from ginger root fragments, for example ginger root powder.

Preferably, the composition according to the invention comprises a ginger root extract.

When it is present in the composition according to the invention, the total content of ginger root extract preferably ranges from 0.02% to 0.2% by weight, more preferentially from 0.05% to 0.2% by weight, relative to the total weight of the composition.

### vi) Solvents

The composition according to the invention may optionally comprise one or more organic solvents other than the hydroxylated compounds iv) described previously.

These organic solvents are preferably chosen from C1-C5 monoalcohols, preferentially C2-C4 monoalcohols. Particularly preferably, ethanol is used.

Preferably, the composition comprises one or more organic solvents other than the hydroxylated compounds iv) described previously, preferentially one or more C2-C4 monoalcohols, better still ethanol.

When it is (they are) present, the total content of organic solvents other than the hydroxylated compounds iv) preferably ranges from 1% to 60% by weight, preferentially from 5% to 50% by weight, more preferentially from 10% to 40% by weight, better still from 20% to 35% by weight, relative to the total weight of the composition.

When the composition comprises ethanol, the ethanol content preferably ranges from 1% to 60% by weight, preferentially from 5% to 50% by weight, more preferentially from 10% to 40% by weight, better still from 20% to 35% by weight, relative to the total weight of the composition.

The composition is preferably aqueous, the water content possibly ranging from 10% to 90% by weight, preferably from 20% to 80% by weight and preferentially from 40% to 70% by weight relative to the total weight of the composition.

vii) Thickening Polymers

The composition according to the invention may optionally comprise one or more thickening polymers.

According to the present invention, the term “thickening polymers” means compounds which, by their presence at a concentration of 0.05% by weight, increase the viscosity of a composition into which they are introduced by at least 20 cps (20 mPa·s), preferably by at least 50 cps (50 mPa·s), at room temperature (25° C.), at atmospheric pressure and at a shear rate of 1 s⁻¹ (the viscosity may be measured using a cone/plate viscometer, a Haake R600 rheometer or the like).

The thickening polymer(s) that may be used in the process according to the invention are preferentially chosen from non-associative thickening polymers bearing sugar units, non-associative thickening polymers without sugar units, associative thickening polymers, and mixtures thereof.

For the purposes of the present invention, the term “sugar unit” means an oxygen-bearing hydrocarbon-based compound containing several alcohol functions, with or without aldehyde or ketone functions, and which includes at least 4 carbon atoms.

The sugar units may be optionally modified by substitution, and/or by oxidation and/or by dehydration.

The sugar units of the thickening polymers are preferably derived from the following sugars: glucose, galactose, arabinose, rhamnose, mannose, xylose, fucose, anhydrogalactose, galacturonic acid, glucuronic acid, mannuronic acid, galactose sulfate, anhydrogalactose sulfate and fructose.

According to the invention, polymers bearing sugar units are also known as polysaccharides.

Non-associative thickening polymers bearing sugar units that may notably be mentioned include native gums such as:
a) tree or shrub exudates, including:
   acacia gum (branched polymer of galactose, arabinose, rhamnose and glucuronic acid);
   ghatti gum (polymer derived from arabinose, galactose, mannose, xylose and glucuronic acid);
   karaya gum (polymer derived from galacturonic acid, galactose, rhamnose and glucuronic acid);
   gum tragacanth (polymer of galacturonic acid, galactose, fucose, xylose and arabinose);
b) gums derived from algae, including:
   agar (polymer derived from galactose and anhydrogalactose);
   alginates (polymers of mannuronic acid and glucuronic acid);
   carrageenans and furcellerans (polymers of galactose sulfate and anhydrogalactose sulfate);
c) gums derived from seeds or tubers, including:
   guar gum (polymer of mannose and galactose);
   locust bean gum (polymer of mannose and galactose);
   fenugreek gum (polymer of mannose and galactose);
   tamarind gum (polymer of galactose, xylose and glucose);
   konjac gum (polymer of glucose and mannose);
d) microbial gums, including:
   xanthan gum (polymer of glucose, mannose acetate, mannose/pyruvic acid and glucuronic acid);
   gellan gum (polymer of partially acylated glucose, rhamnose and glucuronic acid);
   scleroglucan gum (glucose polymer);
e) plant extracts, including:
   cellulose (glucose polymer);
   starch (glucose polymer); and
   inulin.

These polymers may be physically or chemically modified. As physical treatment, mention may notably be made of the temperature.

Chemical treatments that may be mentioned include esterification, etherification, amidation or oxidation reactions. These treatments make it possible to produce polymers that may notably be nonionic, anionic or amphoteric.

Preferably, these chemical or physical treatments are applied to guar gums, locust bean gums, starches and celluloses.

The nonionic guar gums that may be used according to the invention may be modified with C₁-C₆ (poly) hydroxyalkyl groups.

Among the C₁-C₆ (poly) hydroxyalkyl groups, mention may be made, by way of example, of hydroxymethyl, hydroxyethyl, hydroxypropyl and hydroxybutyl groups.

These guar gums are well known from the prior art and may be prepared, for example, by reacting corresponding alkene oxides, for instance propylene oxides, with the guar gum so as to obtain a guar gum modified with hydroxypropyl groups.

The degree of hydroxyalkylation preferably ranges from 0.4 to 1.2 and corresponds to the number of alkylene oxide molecules consumed by the number of free hydroxyl functions present on the guar gum.

Such nonionic guar gums optionally modified with hydroxyalkyl groups are sold, for example, under the trade names Jaguar HP8, Jaguar HP60 and Jaguar HP120 by the company Rhodia Chimie.

The botanical origin of the starch molecules that may be used in the present invention may be cereals or tubers. Thus, the starches are chosen, for example, from corn starch, rice starch, cassava starch, barley starch, potato starch, wheat starch, sorghum starch and pea starch.

The starches may be chemically or physically modified, particularly by one or more of the following reactions: pregelatinization, oxidation, crosslinking, esterification, etherification, amidation, heat treatments.

Distarch phosphates or compounds rich in distarch phosphate will preferentially be used, for instance the product sold under the references Prejel VA-70-T AGGL (gelatinized hydroxypropyl cassava distarch phosphate), Prejel TK1 (gelatinized cassava distarch phosphate) or Prejel 200 (gelatinized acetylated cassava distarch phosphate) by the company Avebe, or Structure Zea from National Starch (gelatinized corn distarch phosphate).

According to the invention, amphoteric starches may also be used, these amphoteric starches comprising one or more anionic groups and one or more cationic groups. The anionic and cationic groups may be bonded to the same reactive site of the starch molecule or to different reactive sites; they are preferably bonded to the same reactive site. The anionic groups may be of carboxylic, phosphate or sulfate type, preferably carboxylic type. The cationic groups may be of primary, secondary, tertiary or quaternary amine type.

The starch molecules may be derived from any plant source of starch, notably such as corn, potato, oat, rice, tapioca, sorghum, barley or wheat. It is also possible to use hydrolysates of the starches mentioned above. The starch is preferably derived from potato.

The non-associative thickening polymers of the invention may be cellulose-based polymers not comprising a C₁₀-C₃₀ fatty chain in their structure.

According to the invention, the term “cellulose-based polymer” refers to any polysaccharide compound bearing in its structure sequences of glucose residues linked together via β-1,4 bonds; in addition to unsubstituted celluloses, the cellulose derivatives may be anionic, cationic, amphoteric or nonionic.

Thus, the cellulose-based polymers that may be used according to the invention may be chosen from unsubstituted celluloses, including those in a microcrystalline form, and cellulose ethers.

Among these cellulose-based polymers, cellulose ethers, cellulose esters and cellulose ether esters are distinguished.

Among the cellulose esters are mineral esters of cellulose (cellulose nitrates, sulfates, phosphates, etc.), organic cellulose esters (cellulose monoacetates, triacetates, amidopropionates, acetatebutyrates, acetatepropionates or acetatetrimellitates, etc.), and mixed organic/mineral esters of cellulose, such as cellulose acetatebutyrate sulfates and cellulose acetatepropionate sulfates. Among the cellulose ester ethers, mention may be made of hydroxypropylmethylcellulose phthalates and ethylcellulose sulfates.

Among the nonionic cellulose ethers not bearing a C₁₀-C₃₀ fatty chain, i.e. “non-associative” cellulose ethers, mention may be made of (C₁-C₄)alkylcelluloses, such as methylcelluloses and ethylcelluloses (for example, Ethocel standard 100 Premium from Dow Chemical); (poly) hydroxy (C₁-C₄)alkylcelluloses, such as hydroxymethylcelluloses, hydroxyethylcelluloses (for example, Natrosol 250 HHR sold by Aqualon) and hydroxypropylcelluloses (for example, Klucel EF from Aqualon); mixed (poly) hydroxy (C₁-C₄)alkyl(C₁-C₄)alkylcelluloses, such as hydroxypropylmethylcelluloses (for example, Methocel E4M from Dow Chemical), hydroxyethylmethylcelluloses, hydroxyethylethylcelluloses (for example, Bermocoll E 481 FQ from Akzo Nobel) and hydroxybutylmethylcelluloses.

Among the anionic cellulose ethers without a fatty chain, mention may be made of (poly) carboxy (C₁-C₄)alkylcelluloses and salts thereof. Mention may be made, by way of example, of carboxymethylcelluloses, carboxymethylmethylcelluloses (for example Blanose 7M from the company Aqualon) and carboxymethylhydroxyethylcelluloses, and the sodium salts thereof.

Among the cationic cellulose ethers without a fatty chain, mention may be made of cationic cellulose derivatives such as cellulose copolymers or cellulose derivatives grafted with a water-soluble quaternary ammonium monomer, and notably described in U.S. Pat. No. 4,131,576, such as (poly) hydroxy (C₁-C₄)alkylcelluloses, for instance hydroxymethyl-, hydroxyethyl- or hydroxypropylcelluloses notably grafted with a methacryloylethyltrimethylammonium, methacrylamidopropyl-trimethylammonium or dimethyldiallylammonium salt. The commercial products corresponding to this definition are more particularly the products sold under the names Celquat® L 200 and Celquat® H 100 by the company National Starch.

Among the non-associative thickening polymers not bearing sugar units that may be used according to the invention, mention may be made of crosslinked acrylic acid or methacrylic acid homopolymers or copolymers, crosslinked 2-acrylamido-2-methylpropanesulfonic acid homopolymers and crosslinked acrylamide copolymers thereof, ammonium acrylate homopolymers, or copolymers of ammonium acrylate and of acrylamide, alone or as mixtures.

A first family of non-associative thickening polymers that is suitable for use is represented by crosslinked acrylic acid homopolymers.

Among the homopolymers of this type, mention may be made of those crosslinked with an allyl alcohol ether of the sugar series, for instance the products sold under the names Carbopol 980, 981, 954, 2984 and 5984 by the company Noveon or the products sold under the names Synthalen M and Synthalen K by the company 3 VSA. These polymers have the INCI name Carbomer.

The non-associative thickening polymers may also be crosslinked (meth)acrylic acid copolymers, such as the polymer sold under the name Aqua SF1 by the company Noveon.

The non-associative thickening polymers may be chosen from crosslinked 2-acrylamido-2-methylpropanesulfonic acid homopolymers and the crosslinked acrylamide copolymers thereof.

Among the partially or totally neutralized crosslinked copolymers of 2-acrylamido-2-methylpropanesulfonic acid and of acrylamide, mention may be made in particular of the product described in Example 1 of EP 503 853, and reference may be made to said document as regards these polymers.

The composition according to the invention may similarly comprise, as non-associative thickening polymers, ammonium acrylate homopolymers or copolymers of ammonium acrylate and of acrylamide.

As examples of ammonium acrylate homopolymers, mention may be made of the product sold under the name Microsap PAS 5193 by the company Hoechst. Among the copolymers of ammonium acrylate and of acrylamide, mention may be made of the product sold under the name Bozepol C Nouveau or the product PAS 5193 sold by the company Hoechst. Reference may notably be made to FR 2 416 723, U.S. Pat. Nos. 2,798,053 and 2,923,692 as regards the description and preparation of such compounds.

Use may also be made of cationic thickening polymers of acrylic type.

Among the thickening polymers, mention may also be made of the associative polymers that are well known to those skilled in the art and notably of nonionic, anionic, cationic or amphoteric nature, preferably anionic.

It is recalled that “associative polymers” are polymers that are capable, in an aqueous medium, of reversibly associating with each other or with other molecules.

Their chemical structure more particularly comprises at least one hydrophilic zone and at least one hydrophobic zone.

“Hydrophobic group” means a radical or polymer with a saturated or unsaturated, linear or branched hydrocarbon-based chain, comprising at least 10 carbon atoms, preferably from 10 to 30 carbon atoms, in particular from 12 to 30 carbon atoms and more preferentially from 18 to 30 carbon atoms.

Preferentially, the hydrocarbon-based group originates from a monofunctional compound. By way of example, the hydrophobic group may be derived from a fatty alcohol such as stearyl alcohol, dodecyl alcohol or decyl alcohol. It may also denote a hydrocarbon-based polymer, for instance polybutadiene.

Among the associative polymers of anionic type, mention may be made of:
(a) those including at least one hydrophilic unit and at least one fatty-chain allyl ether unit, more particularly those whose hydrophilic unit is constituted by an ethylenically unsaturated anionic monomer, even more particularly by a vinylcarboxylic acid and most particularly by an acrylic acid or a methacrylic acid or mixtures thereof.

Among these anionic associative polymers, those that are particularly preferred according to the invention are polymers formed from 20% to 60% by weight of acrylic acid and/or of methacrylic acid, from 5% to 60% by weight of lower alkyl (meth)acrylates, from 2% to 50% by weight of fatty-chain allyl ether, and from 0 to 1% by weight of a crosslinking agent which is a well-known copolymerizable unsaturated polyethylenic monomer, for instance diallyl phthalate, allyl (meth)acrylate, divinylbenzene, (poly)ethylene glycol dimethacrylate or methylenebisacrylamide.

Among the latter polymers, those most particularly preferred are crosslinked terpolymers of methacrylic acid, of ethyl acrylate and of polyethylene glycol (10 EO) stearyl alcohol ether (Steareth-10), notably those sold by the company Ciba under the names Salcare SC 80® and Salcare SC 90®, which are aqueous 30% emulsions of a crosslinked terpolymer of methacrylic acid, of ethyl acrylate and of steareth-10 allyl ether (40/50/10);
(b) those including i) at least one hydrophilic unit of unsaturated olefinic carboxylic acid type, and ii) at least one hydrophobic unit of the type such as a (C₁₀-C₃₀)alkyl ester of an unsaturated carboxylic acid.

(C₁₀-C₃₀)alkyl esters of unsaturated carboxylic acids that are useful in the invention comprise, for example, lauryl acrylate, stearyl acrylate, decyl acrylate, isodecyl acrylate and dodecyl acrylate, and the corresponding methacrylates, lauryl methacrylate, stearyl methacrylate, decyl methacrylate, isodecyl methacrylate and dodecyl methacrylate.

Anionic polymers of this type are described and prepared, for example, according to U.S. Pat. Nos. 3,915,921 and 4,509,949.

Among the anionic associative polymers of this type, use will more particularly be made of those constituted of from 60% to 95% by weight of acrylic acid (hydrophilic unit), 4% to 40% by weight of C₁₀-C₃₀ alkyl acrylate (hydrophobic unit) and 0% to 6% by weight of crosslinking polymerizable monomer, or alternatively those constituted of from 96% to 98% by weight of acrylic acid (hydrophilic unit), 1% to 4% by weight of C₁₀-C₃₀ alkyl acrylate (hydrophobic unit) and 0.1% to 0.6% by weight of crosslinking polymerizable monomer such as those described previously.

Among said polymers above, the ones most particularly preferred according to the present invention are the products sold by the company Goodrich under the trade names Pemulen TR1®, Pemulen TR2®, Carbopol 1382®, and even more preferentially Pemulen TR1®, and the product sold by the company SEPPIC under the name Coatex SX®.

Mention may also be made of the acrylic acid/lauryl methacrylate/vinylpyrrolidone terpolymer sold under the name Acrylidone LM by the company ISP.
(c) maleic anhydride/C₃₀-C₃₈ α-olefin/alkyl maleate terpolymers, such as the product (maleic anhydride/C₃₀-C₃₈ α-olefin/isopropyl maleate copolymer) sold under the name Performa V 1608® by the company Newphase Technologies.
(d) acrylic terpolymers comprising:
i) approximately 20% to 70% by weight of an α,β-monoethylenically unsaturated carboxylic acid [A],
ii) approximately 20% to 80% by weight of an α,β-monoethylenically unsaturated non-surfactant monomer other than [A],
iii) approximately 0.5% to 60% by weight of a nonionic monourethane which is the reaction product of a monohydric surfactant with a monoethylenically unsaturated monoisocyanate,
such as those described in patent application EP-A-0 173 109 and more particularly the terpolymer described in Example 3, namely a methacrylic acid/methyl acrylate/behenyl alcohol dimethyl-meta-isopropenylbenzylisocyanate ethoxylated (40 EO) terpolymer, as an aqueous 25% dispersion.
   (e) copolymers comprising, among their monomers, an α,β-monoethylenically unsaturated carboxylic acid and an ester of an α,β-monoethylenically unsaturated carboxylic acid and of an oxyalkylenated fatty alcohol.

Preferentially, these compounds also comprise, as monomer, an ester of an α,β-monoethylenically unsaturated carboxylic acid and of a C₁-C₄ alcohol.

An example of a compound of this type that may be mentioned is Aculyn 22® sold by the company Röhm & Haas, which is a methacrylic acid/ethyl acrylate/oxyalkylenated stearyl methacrylate terpolymer; and also Aculyn 88, also sold by the company Röhm & Haas. Mention may also be made of Novethix L-10 Polymer sold by the company Lubrizol, having the INCI name Acrylates/Beheneth-25 Methacrylate Copolymer.
(f) amphiphilic polymers including at least one ethylenically unsaturated monomer bearing a sulfonic group, in free or partially or totally neutralized form and comprising at least one hydrophobic part. These polymers may be crosslinked or non-crosslinked. They are preferably crosslinked.

The ethylenically unsaturated monomers bearing a sulfonic group are notably chosen from vinylsulfonic acid, styrenesulfonic acid, (meth)acrylamido (C₁-C₂₂)alkylsulfonic acids, N-(C₁-C22)alkyl (meth)acrylamido (C₁-C22)alkylsulfonic acids such as undecylacrylamidomethanesulfonic acid, and also partially or totally neutralized forms thereof.

More preferentially, use will be made of (meth)acrylamido (C₁-C₂₂)alkylsulfonic acids, for instance acrylamidomethanesulfonic acid, acrylamidoethanesulfonic acid, acrylamidopropanesulfonic acid, 2-acrylamido-2-methylpropanesulfonic acid, methacrylamido-2-methylpropanesulfonic acid, 2-acrylamido-n-butanesulfonic acid, 2-acrylamido-2,4,4-trimethylpentanesulfonic acid, 2-methacrylamidododecylsulfonic acid or 2-acrylamido-2,6-dimethyl-3-heptanesulfonic acid, and also the partially or completely neutralized forms thereof.

Use will more particularly be made of 2-acrylamido-2-methylpropanesulfonic acid (AMPS), and also partially or totally neutralized forms thereof.

The polymers of this family may be chosen notably from random amphiphilic AMPS polymers modified by reaction with a C₆-C₂₂ n-monoalkylamine or di-n-alkylamine, and such as those described in patent application WO 00/31154 (forming an integral part of the content of the description). These polymers may also contain other ethylenically unsaturated hydrophilic monomers chosen, for example, from (meth)acrylic acids, β-substituted alkyl derivatives thereof or esters thereof obtained with monoalcohols or mono- or polyalkylene glycols, (meth)acrylamides, vinylpyrrolidone, maleic anhydride, itaconic acid or maleic acid, or mixtures of these compounds.

The preferred polymers of this family are chosen from amphiphilic copolymers of AMPS and of at least one ethylenically unsaturated hydrophobic monomer.

These same copolymers may also contain one or more ethylenically unsaturated monomers not including a fatty chain, such as (meth)acrylic acids, β-substituted alkyl derivatives thereof or esters thereof obtained with monoalcohols or mono- or polyalkylene glycols, (meth)acrylamides, vinylpyrrolidone, maleic anhydride, itaconic acid or maleic acid, or mixtures of these compounds.

These copolymers are notably described in patent application EP-A 750 899, U.S. Pat. No. 5,089,578 and in the following publications from Yotaro Morishima:
Self-assembling amphiphilic polyelectrolytes and their nanostructures, Chinese Journal of Polymer Science, Vol. 18, No. 40, (2000), 323-336;
Micelle formation of random copolymers of sodium 2-(acrylamido)-2-methylpropanesulfonate and a nonionic surfactant macromonomer in water as studied by fluorescence and dynamic light scattering, Macromolecules, Vol. 33, No. 10 (2000), 3694-3704;
Solution properties of micelle networks formed by non-ionic moieties covalently bound to an polyelectrolyte: salt effects on rheological behavior—Langmuir, Vol. 16, No. 12, (2000) 5324-5332;
Stimuli responsive amphiphilic copolymers of sodium 2-(acrylamido)-2-methylpropanesulfonate and associative macromonomers, Polym. Preprint, Div. Polym. Chem., 40 (2), (1999), 220-221.

Among these polymers, mention may be made of:
crosslinked or non-crosslinked, neutralized or non-neutralized copolymers, including from 15% to 60% by weight of AMPS units and from 40% to 85% by weight of (C₈-C₁₆)alkyl (meth)acrylamide or (C₈-C₁₆)alkyl (meth)acrylate units relative to the polymer, such as those described in patent application EP-A750 899;
terpolymers including from 10 mol % to 90 mol % of acrylamide units, from 0.1 mol % to 10 mol % of AMPS units and from 5 mol % to 80 mol % of n-(C₆-C₁₈)alkylacrylamide units, such as those described in U.S. Pat. No. 5,089,578.

Mention may also be made of copolymers of totally neutralized AMPS and of dodecyl methacrylate, and also crosslinked and non-crosslinked copolymers of AMPS and of n-dodecylmethacrylamide, such as those described in the Morishima articles mentioned above.

Among the cationic associative polymers, mention may be made of:
(a) cationic associative polyurethanes;
(b) the compound sold by the company Noveon under the name Aqua CC and which corresponds to the INCI name Polyacrylate-1 Crosspolymer.

Polyacrylate-1 Crosspolymer is the product of the polymerization of a mixture of monomers comprising:
a di(C₁-C₄ alkyl)amino(C₁-C₆ alkyl) methacrylate,
one or more C₁-C₃₀ alkyl esters of (meth)acrylic acid,
a polyethoxylated C₁₀-C₃₀ alkyl methacrylate (20-25 mol of ethylene oxide units),
a 30/5 polyethylene glycol/polypropylene glycol allyl ether,
a hydroxy (C₂-C₆ alkyl) methacrylate, and
an ethylene glycol dimethacrylate.

(c) quaternized (poly) hydroxyethylcelluloses modified with groups including at least one fatty chain, such as alkyl, arylalkyl or alkylaryl groups including at least 8 carbon atoms, or mixtures thereof. The alkyl radicals borne by the above quaternized celluloses or hydroxyethylcelluloses preferably include from 8 to 30 carbon atoms. The aryl radicals preferably denote phenyl, benzyl, naphthyl or anthryl groups. Examples of quaternized alkylhydroxyethylcelluloses containing C₈-C₃₀ fatty chains that may be indicated include the products Quatrisoft LM 200®, Quatrisoft LM-X 529-18-A®, Quatrisoft LM-X 529-18-B® (C₁₂ alkyl) and Quatrisoft LM-X 529-8® (C₁₈ alkyl) sold by the company Aqualon, and the products Crodacel QM®, Crodacel QL® (C₁₂ alkyl) and Crodacel QS® (C₁₈ alkyl) sold by the company Croda and the product Softcat SL 100® sold by the company Aqualon.

(d) cationic polyvinyllactam polymers.

Such polymers are described, for example, in patent application WO-00/68282.

As cationic poly(vinyllactam) polymers according to the invention, use is particularly made of vinylpyrrolidone/dimethylaminopropylmethacrylamide/dodecyl-dimethylmethacrylamidopropylammonium tosylate terpolymers, vinylpyrrolidone/dimethylaminopropylmethacrylamide/cocoyldimethylmethacryl-amidopropylammonium tosylate terpolymers, vinylpyrrolidone/dimethylamino-propylmethacrylamide/lauryldimethylmethacrylamidopropylammonium tosylate or chloride terpolymers.

The amphoteric associative polymers are preferably chosen from those including at least one noncyclic cationic unit. Even more particularly, preference is given to those prepared from or comprising 1 to 20 mol %, preferably 1.5 to 15 mol % and even more particularly 1.5 to 6 mol % of fatty-chain monomer relative to the total number of moles of monomers.

Amphoteric associative polymers according to the invention are described and prepared, for example, in patent application WO 98/44012.

Among the amphoteric associative polymers according to the invention, preference is given to acrylic acid/(meth)acrylamidopropyltrimethylammonium chloride/stearyl methacrylate terpolymers.

The associative polymers of nonionic type that may be used according to the invention are preferably chosen from:

(a) copolymers of vinylpyrrolidone and of fatty-chain hydrophobic monomers, of which mention may be made, by way of example:
the products Antaron V216® or Ganex V216® (vinylpyrrolidone/hexadecene copolymer), sold by the company ISP;
   the products Antaron V220® or Ganex V220® (vinylpyrrolidone/eicosene copolymer), sold by the company ISP;
(b) copolymers of C₁-C₆ alkyl methacrylates or acrylates and of amphiphilic monomers including at least one fatty chain, for instance the oxyethylenated methyl acrylate/stearyl acrylate copolymer sold by the company Goldschmidt under the name Antil 208®;
(c) copolymers of hydrophilic methacrylates or acrylates and of hydrophobic monomers including at least one fatty chain, for instance the polyethylene glycol methacrylate/lauryl methacrylate copolymer.
(d) polyurethane polyethers including in their chain both hydrophilic blocks usually of polyoxyethylenated nature and hydrophobic blocks, which may be aliphatic sequences alone and/or cycloaliphatic and/or aromatic sequences;
(e) polymers with an aminoplast ether backbone containing at least one fatty chain, such as the Pure Thix® compounds sold by the company Sud-Chemie;
(f) celluloses or derivatives thereof, modified with groups including at least one fatty chain, such as alkyl, arylalkyl or alkylaryl groups or mixtures thereof in which the alkyl groups are of C₈, and in particular:
   nonionic alkylhydroxyethylcelluloses such as the products Natrosol Plus Grade 330 CS and Polysurf 67 (C₁₆ alkyl) sold by the company Aqualon;
   nonionic nonoxynylhydroxyethylcelluloses such as the product Amercell HM-1500 sold by the company Amerchol;
   nonionic alkylcelluloses, such as the product Bermocoll EHM 100 sold by the company Berol Nobel;
(g) associative guar derivatives, for instance hydroxypropyl guars modified with a fatty chain, such as the product Esaflor HM 22 (modified with a C₂₂ alkyl chain) sold by the company Lamberti; the product Miracare XC 95-3 (modified with a C₁₄ alkyl chain) and the product RE 205-146 (modified with a C₂₀ alkyl chain) sold by Rhodia Chimie.

Preferably, the polyurethane polyethers include at least two hydrocarbon-based lipophilic chains containing from 6 to 30 carbon atoms, separated by a hydrophilic block, the hydrocarbon-based chains possibly being side chains or chains at the end of the hydrophilic block. In particular, it is possible for one or more pendent chains to be envisaged. In addition, the polymer may include a hydrocarbon-based chain at one end or at both ends of a hydrophilic block.

The polyurethane polyethers may be multiblock, in particular in triblock form. The hydrophobic blocks may be at each end of the chain (for example: triblock copolymer bearing a hydrophilic central block) or distributed both at the ends and in the chain (for example, multiblock copolymer). These same polymers may also be graft polymers or star polymers.

The fatty-chain nonionic polyurethane polyethers may be triblock copolymers, the hydrophilic block of which is a polyoxyethylenated chain including from 50 to 1000 oxyethylene groups. The nonionic polyurethane polyethers include a urethane bond between the hydrophilic blocks, whence arises the name.

By extension, also included among the fatty-chain nonionic polyurethane polyethers are those in which the hydrophilic blocks are linked to the lipophilic blocks via other chemical bonds.

As examples of nonionic fatty-chain polyurethane polyethers that may be used in the invention, use may also be made of Rheolate 205® bearing a urea function, sold by the company Rheox, or Rheolate® 208, 204 or 212, and also Acrysol RM 184®.

Mention may also be made of the product Elfacos T210® bearing a C₁₂-C₁₄ alkyl chain, and the product Elfacos T212® bearing a C₁₈ alkyl chain, from Akzo.

The product DW 1206B® from Röhm & Haas bearing a C₂₀ alkyl chain and a urethane bond, provided at a solids content of 20% in water, may also be used.

Use may also be made of solutions or dispersions of these polymers, notably in water or in an aqueous/alcoholic medium. Examples of such polymers that may be mentioned include Rheolate® 255, Rheolate® 278 and Rheolate® 244 sold by the company Rheox. Use may also be made of the products DW 1206F and DW 1206J sold by the company Rohm & Haas.

The polyurethane polyethers that may be used according to the invention are in particular those described in the article by G. Fonnum, J. Bakke and Fk. Hansen-Colloid Polym. Sci., 271, 380-389 (1993).

It is even more particularly preferred to use a polyurethane polyether that may be obtained by polycondensation of at least three compounds comprising (i) at least one polyethylene glycol comprising from 150 to 180 mol of ethylene oxide, (ii) stearyl alcohol or decyl alcohol, and (iii) at least one diisocyanate.

Such polyurethane polyethers are sold notably by the company Röhm & Haas under the names Aculyn 46® and Aculyn 44® [Aculyn 46® is a polycondensate of polyethylene glycol containing 150 or 180 mol of ethylene oxide, of stearyl alcohol and of methylenebis(4-cyclohexyl isocyanate) (SMDI), at 15% by weight in a matrix of maltodextrin (4%) and water (81%); Aculyn 44® is a polycondensate of polyethylene glycol containing 150 or 180 mol of ethylene oxide, of decyl alcohol and of methylenebis(4-cyclohexyl isocyanate) (SMDI), at 35% by weight in a mixture of propylene glycol (39%) and water (26%)].

Preferably, the composition comprises one or more thickening polymers, preferentially chosen from non-associative thickening polymers free of sugar units, anionic associative thickening polymers, in particular anionic associative acrylic polymers, and mixtures thereof.

Preferably, when the thickening polymer(s) are present in the composition according to the invention, the total content of thickening polymer(s) is in the range from 0.05% to 4% by weight, more preferentially from 0.1% to 2% by weight, even more preferentially from 0.2% to 1% by weight, and better still from 0.3% to 0.8% by weight, relative to the total weight of the composition.

### Additives

The composition may optionally also comprise one or more additives other than the compounds described previously, such as those chosen from cationic, anionic, amphoteric or zwitterionic, nonionic surfactants and mixtures thereof, cationic, anionic, nonionic, amphoteric polymers or mixtures thereof, anti-dandruff agents, anti-seborrhoeic agents, vitamins and pro-vitamins including panthenol, sunscreens, sequestrants, plasticizers, solubilizing agents, acidifying agents, opacifying or nacreous agents, antioxidants, hydroxy acids, fragrances, preserving agents and ceramides.

Needless to say, a person skilled in the art will take care to select this or these optional additional compounds such that the advantageous properties intrinsically associated with the composition according to the invention are not, or are not substantially, adversely affected by the envisaged addition(s).

The above additives may generally be present in an amount, for each of them, of between 0 and 20% by weight relative to the total weight of the composition.

### Process

The invention also relates to a process for the cosmetic treatment of human keratin materials such as the skin, notably human scalp, hair and/or eyelashes, comprising at least the application of the cosmetic composition as defined previously.

The composition according to the present invention is preferably applied topically, notably to the scalp and/or the hair.

The application of the composition may or may not be followed by rinsing after a possible leave-on time.

Preferably, the application of the composition according to the invention is not followed by rinsing.

Preferably, the cosmetic treatment process is a process for caring for the hair and/or the scalp.

A subject of the present invention is also the use of a cosmetic composition as defined previously for treating the loss and/or promoting the regrowth of keratin fibres.

A subject of the present invention is also the use of a cosmetic composition as defined previously for increasing the diameter of keratin fibres and/or reinforcing keratin fibres against breakage.

The examples that follow serve to illustrate the invention without, however, being limiting in nature.

### EXAMPLES

In the examples that follow, all the amounts are given as mass percentages of active material (AM) relative to the total weight of the composition (unless otherwise mentioned).

### a) Compositions

Compositions A to D according to the present invention and comparative composition E were prepared using the ingredients whose contents are indicated in the tables below:

TABLE 1

| | Composition | Composition | Composition | Composition | Composition |
|---|---|---|---|---|---|
| | A | B | C | D | E |
| | (invention) | (invention) | (invention) | (invention) | (comparative) |
| Propylene glycol | 2.5 | 2.5 | 2.5 | 2.5 | — |
| Niacinamide | 3 | 3.5 | 3 | 3 | 3 |
| Ginger root | 0.1 | 0.1 | 0.1 | 0.1 | 0.1 |
| extract | | | | | |
| Diaminopyrimidine | 1.5 | 1.5 | 1.5 | 1.5 | 1.5 |
| oxide | | | | | |
| Piroctone | 0.25 | 0.25 | 0.25 | 0.25 | 0.25 |
| olamine | | | | | |
| Acrylates/ | 0.45 | 0.53 | 0.6 | 0.45 | 0.6 |
| Beheneth-25 | | | | | |
| Methacrylate | | | | | |
| Copolymer | | | | | |
| PEG-40 castor oil | 0.1 | 0.1 | 0.1 | 0.3 | 0.1 |
| Ethanol | 27 | 37.5 | 37.5 | 30 | 40 |
| Water | q.s. for 100 | q.s. for 100 | q.s. for 100 | q.s. for 100 | q.s. for 100 |

### b) Protocol

A test was carried out by expert hairstylists with consumers. The test makes it possible to test two formulations per consumer, each product being applied to one half of the head.

The first day, a shampoo wash is performed in order to clean the scalp by removing any residue of product that might have been applied beforehand by the consumer. The head of hair and the scalp are partly dried with a towel.

A first application of 3 mL of product to one half of the scalp is then carried out. The scalp is observed. The scalp and the hair are then dried and the scalp is observed.

The next day, the consumers' scalps are observed on their return. A second application of 3 mL of product to the same half of the scalp is then carried out. The scalp is observed.

Among the various parameters observed, the powdering (appearance of powder) is noted by the hairstylists and the degree of powdering is judged to be acceptable or unacceptable by the hairstylists who are experts in sensory properties.

### c) Results

The results are presented in the table below:

TABLE 2

| | 1^{st} application, | | 2^{nd} |
|---|---|---|---|
| Composition | dry hair | Return | application |
| Composition | No powdering | No powdering | No powdering |
| A | | | |
| Composition | No powdering | No powdering | No powdering |
| B | | | |
| Composition | No powdering | No powdering | No powdering |
| C | | | |
| Composition | No powdering | No powdering | No powdering |
| D | | | |
| Composition | Powdering | Powdering | Powdering |
| E | | | |

Compositions A to D according to the invention do not leave any powdery effect (presence of particles) on the scalp or the hair after drying the composition according to the invention.

The same result is observed after renewed application of the compositions after 24 h, or a very mild powdery effect is observed that is judged to be acceptable by a hairstylist who is an expert in scalp cleanliness.

Comparative composition E leaves a powdery effect on the scalp and the hair.

1-11. (canceled)

12. A cosmetic composition comprising:
i) 2,4-diaminopyrimidine 3-N-oxide, an organic or mineral acid salt thereof or a hydrate thereof,
ii) piroctone olamine,
iii) niacinamide, and
iv) at least one hydroxylated compound with a melting point below 25° C. at atmospheric pressure (1.013×10⁵ Pa) and a boiling point of greater than or equal to 170° C., wherein the total amount of the at least one hydroxylated compound is at least 1% by weight, relative to the total weight of the composition.

13. The composition according to claim 12, wherein the total amount of i) 2,4-diaminopyrimidine 3-N-oxide, an organic or mineral acid salt thereof, or a hydrate thereof ranges from 0.05% to 8% by weight, relative to the total weight of the composition.

14. The composition according to claim 12, wherein the total amount of ii) piroctone olamine ranges from 0.05% to 2% by weight, relative to the total weight of the composition.

15. The composition according to claim 12, wherein the total amount of iii) niacinamide ranges from 1% to 5% by weight, relative to the total weight of the composition.

16. The composition according to claim 12, wherein the at least one hydroxylated compound iv) is chosen from linear C2-C8 diols, aromatic C6-C8 monoalcohols or mixtures thereof.

17. The composition according to claim 12, wherein the total amount of the at least one hydroxylated compound iv) ranges from 1% to 15% by weight, relative to the total weight of the composition.

18. The composition according to claim 12, further comprising ginger root extract in a total amount ranging from 0.02% to 0.2% by weight, relative to the total weight of the composition.

19. The composition according to claim 12, further comprising at least one thickening polymer chosen from non-associative thickening polymers free of sugar units, anionic associative thickening polymers, or mixtures thereof.

20. A method for treating human keratin materials, comprising applying to said keratin materials a composition comprising:
i) 2,4-diaminopyrimidine 3-N-oxide, an organic or mineral acid salt thereof or a hydrate thereof,
ii) piroctone olamine,
iii) niacinamide, and
iv) at least one hydroxylated compound with a melting point below 25° C. at atmospheric pressure (1.013×10⁵ Pa) and a boiling point of greater than or equal to 170° C., wherein the total amount of the at least one hydroxylated compound is at least 1% by weight, relative to the total weight of the composition.

21. The method for treating human keratin materials according to claim 20, wherein the method is for treating loss and/or promoting regrowth of keratin fibres.

22. The method for treating human keratin materials according to claim 20, wherein the method is for increasing the diameter of keratin fibres and/or reinforcing keratin fibres against breakage.
